(19) United States
(12) Patent Application Publication
(10) Pub. No.: US 2014/0119987 A1
(43) Pub. Date: May 1, 2014

(54) METHOD OF FABRICATING MEDICAL IMPLANTS

(71) Applicant: Takahiro OGAWA, Torrance, CA (US)

(72) Inventor: Takahiro OGAWA, Torrance, CA (US)

(73) Assignee: The Regents of the University of California, Oakland, CA (US)

(21) Appl. No.: 14/150,661

(22) Filed: Jan. 8, 2014

Related U.S. Application Data
(63) Continuation of application No. PCT/US12/45619, filed on Jul. 5, 2012.
(60) Provisional application No. 61/505,876, filed on Jul. 8, 2011.

(51) Int. Cl. A61L 2/10; A61F 2/28

(52) U.S. Cl. CPC A61L 2/10; A61F 2/28
USPC 422/23; 623/16.11; 422/24; 250/455.11

(57) ABSTRACT

Provided herein is methods of fabricating a medical implant and methods of using the same.

### RELATED APPLICATION

This application claims priority to U.S. Provisional Application 61/505,876 filed on Jul. 8, 2011 and entitled “NOVEL WAY TO ACTIVATE IMPLANT MATERIALS,” which is hereby incorporated by reference herein in its entirety.

### BACKGROUND OF THE INVENTION

1. Field of the Invention

This invention generally relates to a medical implant for biomedical use. In particular, the present invention relates to methods of activating medical implant materials.

2. Description of the Background

Restoration of skeletal defects or wounds such as femoral neck fracture and spine fusion is a common procedure. For example, over 500,000 and 250,000 procedures are performed annually in the U.S. for hip prosthesis medical implantation and spine fusion surgery, respectively. Meanwhile, about 74 million people in the U.S., which amounts to about 30% of adult population in the U.S., have at least one qradrant of posterior missing tooth that needs to be restored.

Some metallic materials such as titanium are proven biocompatible materials. For example, use of titanium medical implants has become a standard treatment to replace missing teeth and to fix diseased, fractured or transplanted bone. Restorative treatment of missing teeth using dental medical implants such as titanium medical implants have considerable oral health impact, by which masticatory function (Carlsson G E, Lindquist L W, Int. J. Prosthodont 7(5):448-53 (1994); Geertman M E, et al., Community Dent Oral Epidemiol 24(1):79-84 (1996); Pera P, et al., J Oral Rehabil 25(6):462-7 (1998); van Kampen F M, et al., J Dent Res 83(9):708-11 (2004)), Speech (Heydecke G, et al., J Dent Res 83(3):236-40 (2004)) and daily performance and quality of life (Melas F, et al., Int J Oral Maxillofac Medical implants 16(5):700-12 (2001)) are improved, when compared to the conventional removable denture treatment. In treatments of facial defect resulting from cancer or injury, the use of endosseous medical implants is crucial to retain the prosthesis (Roumanas E D, et al., Int J Prosthodont 15(4):325-32 (2002)). However, the application of medical implant therapy in these fields is still limited because of various risk factors including anatomy and quality of host bone (van Steenberghe D, et al., Clin Oral Medical implants Res 13(6):617-22 (2002)), systemic conditions including diabetes (Nevins M L, Int J Oral Maxillofac Medical implants 13(5):620-9 (1998); Takeshita F, et al., J Periodontol 69(3):314-20 (1998) and osteoporosis (Ozawa S, et al., Bone 30(1):137-43 (2002)), and ageing (Takeshita F, et al., J Biomed Mater Res 34(1):1-8 (1997)). More importantly, long healing time (about 4-10 months) required for titanium medical implants to integrate with surrounding bone restricts the application of this beneficial treatment. For example, in the U.S., dental medical implant therapy has penetrated into only 2% of the potential patients.

In the orthopedic field, the restoration of femoral neck fracture or spine fusion, for example, is a common problem. For example, of over 250,000 procedures performed annually in the U.S. for spine fusion surgery, about 30% or more of patients fail to achieve a solid bony union. The nature and location of bone fracture at these areas do not allow for bone immobilization (e.g., cast splinting) for better healing.

Despite the growing needs of titanium medical implants, a decent percentage of unsuccessful medical implants, for instance, ranging 5%-40% in orthopedic medical implants[2-5], and limited application due to unfavorable host site anatomy [6-10], and protracted healing time of medical implants, particularly in dental medical implants, are the immediate challenges. Furthermore, the medical implant placement, facing often times the impaired bone regenerative potential, such as osteoporotic and aged metabolic properties, increase the level of difficulty to achieve the biological requirements of bone-titanium integration[7, 9-11]. Therefore, technologies to enhance the bioactivity of titanium surfaces are desired.

Regardless of the use in dental and orthopedic therapy, medical implant products are sold in the storable device in a sterilized package. During the inventory, transportation, and circulation, the medical implant products are advertently and unavoidably in the low-temperature condition (lower than room temperature, such as 25° C.). The medical implant products are also often exposed in low temperature during the storage at the peripheral user levels, such as in the dental office and orthopedic hospital. Medical implant products in various sizes and types need to be in stock for various purposes and indications. Thus, the drastic temperature change is a nearly unavoidable atmospheric change for medical implant products in the current medical and commercial system. It is practically unlikely for medical implant products to be delivered without being exposed in the temperature lower than the regular room temperature.

For successful treatment outcome of dental and orthopedic medical implants, the medical implants need to integrate with newly formed bone or surrounding bone to generate sufficient anchoring capacity. As mentioned above, the phenomenon is called bone-medical implant integration or osseointegration. To ensure the successful bone-medical implant integration, it is essential that bone-making cells, such as osteoblasts, osteoprogenitor cells, or stem cells, need to attach and adhere to medical implant surfaces. UV light treatment has been used for medical purpose because of its bacteriocidal ability. However, prior methods fail to recognize the low medical implant temperature plays a critical role in the failure of medical implant osseointegration and address such issue. The embodiments described below address the above identified issues and needs.

### SUMMARY OF THE INVENTION

In one aspect, it is provided a method of fabricating a medical implant, comprising treating a medical implant by ultraviolet light (UV) in a closed environment,

causing the temperature of the medical implant to be between room temperature (Rt) and about 37° C.,

wherein the treating and causing acts are performed immediately prior to placing the medical implant in a site in need thereof in a subject.

In some embodiments of the method, the medical implant has a temperature or is exposed to a temperature below room temperature (Rt) or above body temperature prior to the UV treatment.

In some embodiments of the method, the medical implant has a temperature or is exposed to a temperature between 0° C. and below Rt (e.g., about 20° C.) prior to receiving UV treatment.

In some embodiments of the method, the medical implant has a temperature or is exposed to a temperature of 40° C. or above prior to receiving the UV treatment.

In some embodiments of the method, causing the temperature of the medical implant to be between room temperature (Rt) and about 37° C. comprises the act of heating (e.g., heating by the UV treatment) or cooling.

In some embodiments of the method, optionally in combination with any or all of the various embodiments above, the closed environment is a closed chamber.

In some embodiments of the method, optionally in combination with any or all of the various embodiments above, the closed environment is a closed chamber filled with an inert gas, clean air, or carbon-free air.

In some embodiments of the method, optionally in combination with any or all of the various embodiments above, the inert gas comprises N₂, He, or Ar.

In some embodiments of the method, optionally in combination with any or all of the various embodiments above, the medical implant comprises a metallic material.

In some embodiments of the method, optionally in combination with any or all of the various embodiments above, medical implant comprises a surface comprising a micro or nanostructures.

In some embodiments of the method, optionally in combination with any or all of the various embodiments above, the metallic material comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

In some embodiments of the method, optionally in combination with any or all of the various embodiments above, the medical implant is selected from the group consisting of tooth medical implants, jaw bone medical implant, repairing and stabilizing screws, pins, frames (e.g., mesh frames), and plates for bone, spinal medical implants, femoral medical implants, neck medical implants, knee medical implants, wrist medical implants, joint medical implants such as an artificial hip joint, maxillofacial medical implants such as ear and nose medical implants, limb prostheses for conditions resulting from injury and disease, and combinations thereof.

In some embodiments of the method, optionally in combination with any or all of the various embodiments above, the medical implant comprises a non-metallic material.

In some embodiments of the method, optionally in combination with any or all of the various embodiments above, the non-metallic material comprises a polymeric material or a bone cement material.

In some embodiments of the method, optionally in combination with any or all of the various embodiments above, the bone cement material comprises a material selected from the group consisting of polyacrylates, polyesters, bioglass, ceramics, calcium-based materials, calcium phosphate-based materials, and combinations thereof.

In some embodiments of the method, optionally in combination with any or all of the various embodiments above, bone cement material comprises poly(methyl methacrylate) (PMMA) or methyl methacrylate (MMA).

In a second aspect, it is provided a system, comprising a chamber element (100), an ultraviolet light (UV) element (200), and a medical implant element (300);

wherein the chamber element (100) forms a closed environment that houses the medical implant element (300) and the UV element (200),

wherein the medical implant element (300) comprises a medical implant having a temperature or is exposed to a temperature of below room temperature (Rt) (e.g., from 0° C. to about 20° C.) or above body temperature prior to receiving UV treatment, and

wherein, in the chamber element (100), the medical implant receives UV treatment and is caused to have a temperature between the room temperature and about 37° C.

In some embodiments of the system, optionally in combination with any or all of the various embodiments above, the system further comprises a timing element (400) and a thermometer (500).

In some embodiments of the system, optionally in combination with any or all of the various embodiments above, the system further comprises inert gas, clean air, or carbon-free air (600).

In some embodiments of the system, optionally in combination with any or all of the various embodiments above, the medical implant has a temperature or is exposed to a temperature of about 40° C. or above prior to receiving the UV treatment.

In some embodiments of the system, optionally in combination with any or all of the various embodiments above, the medical implant has a temperature or is exposed to a temperature from about 0° C. to about 20° C. prior to receiving the UV treatment.

In some embodiments of the system, optionally in combination with any or all of the various embodiments above, the medical implant is selected from the group consisting of tooth medical implants, jaw bone medical implant, repairing and stabilizing screws, pins, frames (e.g., mesh frames), and plates for bone, spinal medical implants, femoral medical implants, neck medical implants, knee medical implants, wrist medical implants, joint medical implants such as an artificial hip joint, maxillofacial medical implants such as ear and nose medical implants, limb prostheses for conditions resulting from injury and disease, and combinations thereof.

### BRIEF DESCRIPTION OF THE DRAWINGS

FIG. 1 shows the test results on cell adhesion to three different titanium disks at different temperatures.

FIG. 2 shows the test results on cell adhesion to three different titanium disks stored in water at different temperatures.

FIG. 3 shows the test results on cell attraction capability following UV treatment of cold titanium disks.

FIG. 4 shows that UV treatment was also effective on CoCr alloy to recover its temperature and cell attraction capability.

FIG. 5 shows that UV treatment of cold titanium is more effective under closed conditions than in open conditions

FIG. 6 shows an embodiment of a system of invention provided herein.

### DETAILED DESCRIPTION

In one aspect, it is provided a method of fabricating a medical implant, comprising treating a medical implant by ultraviolet light (UV) in a closed environment, causing the temperature of the medical implant to be between room temperature (Rt) and about 37° C.,

wherein the treating and causing acts are performed immediately prior to placing the medical implant in a site in need thereof in a subject.

In some embodiments of the method, the medical implant has a temperature or is exposed to a temperature below room temperature (Rt) or above body temperature prior to the UV treatment.

In some embodiments of the method, the medical implant has a temperature or is exposed to a temperature between 0° C. and below Rt (e.g., about 20° C.) prior to receiving UV treatment.

In some embodiments of the method, the medical implant has a temperature or is exposed to a temperature of 40° C. or above prior to receiving the UV treatment.

In some embodiments of the method, causing the temperature of the medical implant to be between room temperature (Rt) and about 37° C. comprises the act of heating (e.g., heating by the UV treatment) or cooling.

In some embodiments of the method, optionally in combination with any or all of the various embodiments above, the closed environment is a closed chamber.

In some embodiments of the method, optionally in combination with any or all of the various embodiments above, the closed environment is a closed chamber filled with an inert gas, clean air, or carbon-free air.

In some embodiments of the method, optionally in combination with any or all of the various embodiments above, the inert gas comprises N₂, He, or Ar.

In some embodiments of the method, optionally in combination with any or all of the various embodiments above, the medical implant comprises a metallic material.

In some embodiments of the method, optionally in combination with any or all of the various embodiments above, medical implant comprises a surface comprising a micro or nanostructures.

In some embodiments of the method, optionally in combination with any or all of the various embodiments above, the metallic material comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

In some embodiments of the method, optionally in combination with any or all of the various embodiments above, the medical implant is selected from the group consisting of tooth medical implants, jaw bone medical implant, repairing and stabilizing screws, pins, frames (e.g., mesh frames), and plates for bone, spinal medical implants, femoral medical implants, neck medical implants, knee medical implants, wrist medical implants, joint medical implants such as an artificial hip joint, maxillofacial medical implants such as ear and nose medical implants, limb prostheses for conditions resulting from injury and disease, and combinations thereof.

In some embodiments of the method, optionally in combination with any or all of the various embodiments above, the medical implant comprises a non-metallic material.

In some embodiments of the method, optionally in combination with any or all of the various embodiments above, the non-metallic material comprises a polymeric material or a bone cement material.

In some embodiments of the method, optionally in combination with any or all of the various embodiments above, the bone cement material comprises a material selected from the group consisting of polyacrylates, polyesters, bioglass, ceramics, calcium-based materials, calcium phosphate-based materials, and combinations thereof.

In some embodiments of the method, optionally in combination with any or all of the various embodiments above, bone cement material comprises poly(methyl methacrylate) (PMMA) or methyl methacrylate (MMA).

In a second aspect, it is provided a system, comprising a chamber element (100), an ultraviolet light (UV) element (200), and a medical implant element (300);

wherein the chamber element (100) forms a closed environment that houses the medical implant element (300) and the UV element (200),

wherein the medical implant element (300) comprises a medical implant having a temperature or is exposed to a temperature of below room temperature (Rt) (e.g., from 0° C. to about 20° C.) or above body temperature prior to receiving UV treatment, and

wherein, in the chamber element (100), the medical implant receives UV treatment and is caused to have a temperature between the room temperature and about 37° C.

In some embodiments of the system, optionally in combination with any or all of the various embodiments above, the system further comprises a timing element (400) and a thermometer (500).

In some embodiments of the system, optionally in combination with any or all of the various embodiments above, the system further comprises inert gas, clean air, or carbon-free air (600).

In some embodiments of the system, optionally in combination with any or all of the various embodiments above, the medical implant has a temperature or is exposed to a temperature of about 40° C. or above prior to receiving the UV treatment.

In some embodiments of the system, optionally in combination with any or all of the various embodiments above, the medical implant has a temperature or is exposed to a temperature from about 0° C. to about 20° C. prior to receiving the UV treatment.

In some embodiments of the system, optionally in combination with any or all of the various embodiments above, the medical implant is selected from the group consisting of tooth medical implants, jaw bone medical implant, repairing and stabilizing screws, pins, frames (e.g., mesh frames), and plates for bone, spinal medical implants, femoral medical implants, neck medical implants, knee medical implants, wrist medical implants, joint medical implants such as an artificial hip joint, maxillofacial medical implants such as ear and nose medical implants, limb prostheses for conditions resulting from injury and disease, and combinations thereof.

As used herein, the term treating with an ultraviolet light “UV” can be used interchangeably with the term “light activation,” “light radiation,” “light irradiation,” “UV light activation,” “UV light radiation,” or “UV light irradiation.”

As used herein, the term “UV” or “UV light” shall not encompass a UV laser or UV laser beam. Such UV light does not encompass any UV beam obtained through optical amplification such as those fall within the definition of laser as described in Gould, R. Gordon (1959). “The LASER, Light Amplification by Stimulated Emission of Radiation”. In Franken, P. A. and Sands, R. H. (Eds.). The Ann Arbor Conference on Optical Pumping, the University of Michigan, 15 Jun. through 18 Jun. 1959. p. 128.

As used herein, the term room temperature or Rt generally refers to a temperature of about 25° C. In some embodiments, the term Rt refers to a temperature of 25±1° C.

As used herein, the term body temperature generally refers to a temperature of about 37° C. In some embodiments, the term Rt refers to a temperature from 36° C. to 37.5° C.

As used herein, the term “significantly below room temperature” refers to a temperature of about 20° C. or below, e.g., 0° C., 5° C., 10° C., or 15° C.

As used herein, the term “significantly above room temperature” refers to a temperature of above body temperature, e.g., 38° C., 40° C., 45° C., 50° C., or 55° C.

As used herein, the term “carbon-free air” refers to an air environment that is free from any carbon content or substantially free from any carbon content. Substantially free from any carbon content shall mean an air environment that is removed of at least 90% carbon content (as compared to a normal air environment), which can also be referred to as carbon-minimum air. As used herein, the term “carbon content” refers to any contamination in air containing carbon that is not carbon dioxide. Such contamination can be any organic species, carbon particles, or an inorganic compound in the air that contains carbon.

As used herein, the term “storage in liquid” generally refers to a liquid storage medium for commonly used for storage of medical implants, for example, water or ddH₂O.

### Osteophilic Surface

The term “osteophilic surface” refers to a surface that imparts enhanced tissue integration capabilities to a medical implant. An osteophilic surface can include hydroxyl groups, oxides or both and can have micro or nanostructurs. In some embodiments, the nanostructures can include nanoconstructs such as nanospheres, nanocones, nanopyramids, other nanoconstructs or combinations thereof. In some embodiments, the micro or nanoconstructs have a size in the range between about 1 nm and about 1000 μm, about 1 nm and about 400 μm, about 1 nm and about 100 μm, about 1 nm and about 40 μm, about 1 nm and about 10 μm, about 1 nm and about 1000 nm, about 1 nm and about 400 nm, between about 1 nm and about 200 nm, between about 1 nm and about 100 nm, between about 10 nm and about 100 nm, between about 10 nm and about 70 nm, between about 20 nm and about 40 nm or between about 20 nm and about 40 nm.

As used herein, the term “tissue integration capability” refers to the ability of a medical implant to be integrated into the tissue of a biological body. The tissue integration capability of a medical implant can be generally measured by several factors, one of which is wettability of the medical implant surface, which reflects the hydrophilicity/oleophilicty (hydrophobicity), or hemophilicity of a medical implant surface. Hydrophilicity and oleophilicity are relative terms and can be measured by, e.g., water contact angle (Oshida Y, et al., J Mater Science 3:306-312 (1992)), and area of water spread (Gifu-kosen on line text, http://www.gifu-nct.ac.jp/elec/tokoro/fft/contact-angle.html). For purposes of the present invention, the hydrophilicity/oleophilicity can be measured by contact angle or area of water spread of a medical implant surface described herein relative to the ones of the control medical implant surfaces. Relative to the medical implant surfaces not treated with the process described herein, a medical implant treated with the process described herein has a substantially lower contact angle or a substantially higher area of water spread.

### Medical Implants

The medical implants described herein with enhanced tissue integration capabilities include any medical implants currently available in medicine or to be introduced in the future. The medical implants can be metallic or non-metallic medical implants. Non-metallic medical implants include, for example, ceramic medical implants, calcium phosphate or polymeric medical implants. Useful polymeric medical implants can be any biocompatible medical implants, e.g., bio-degradable polymeric medical implants. Representative ceramic medical implants include, e.g., bioglass and silicon dioxide medical implants. Calcium phosphate medical implants includes, e.g., hydroxyapatite, tricalcium phosphate (TCP). Exemplary polymeric medical implants include, e.g., poly-lactic-co-glycolic acid (PLGA), polyacrylate such as polymethacrylates and polyacrylates, and poly-lactic acid (PLA) medical implants. In some embodiments, the medical implant described herein can specifically exclude any of the aforementioned materials.

In some embodiments, the medical implant comprises a metallic medical implant and a bone-cement material. The bone cement material can be any bone cement material known in the art. Some representative bone cement materials include, but are not limited to, polyacrylate or polymethacrylate based materials such as poly(methyl methacrylate) (PMMA)/methyl methacrylate (MMA), polyester based materials such as PLA or PLGA, bioglass, ceramics, calcium phosphate-based materials, calcium-based materials, and combinations thereof. In some embodiments, the medical implant can include any polymer described below. In some embodiments, the medical implant described herein can specifically exclude any of the aforementioned materials.

The metallic medical implants described herein include titanium medical implants and non-titanium medical implants. Titanium medical implants include tooth or bone replacements made of titanium or an alloy that includes titanium. Titanium bone replacements include, e.g., knee joint and hip joint prostheses, femoral neck replacement, spine replacement and repair, neck bone replacement and repair, jaw bone repair, fixation and augmentation, transplanted bone fixation, and other limb prostheses. None-titanium metallic medical implants include tooth or bone medical implants made of gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, e.g., stainless steel, or combinations thereof. Some examples of alloys are titanium-nickel allows such as nitanol, chromium-cobalt alloys, stainless steel, or combinations thereof. In some embodiments, the metallic medical implant can specifically exclude any of the aforementioned metals.

The medical implant described herein can be porous or non-porous medical implants. Porous medical implants can impart better tissue integration while non-porous medical implants can impart better mechanical strength.

The medical implants can be metallic medical implants or non-metallic medical implants. In some embodiments, the medical implants are metallic medical implants such as titanium medical implants, e.g., titanium medical implants for replacing missing teeth (dental medical implants) or fixing diseased, fractured or transplanted bone. Other exemplary metallic medical implants include, but are not limited to, titanium alloy medical implants, chromium-cobalt alloy medical implants, platinum and platinum alloy medical implants, nickel and nickel alloy medical implants, stainless steel medical implants, zirconium, chromium-cobalt alloy, gold or gold alloy medical implants, and aluminum or aluminum alloy medical implants.

The medical implants provided herein can be subjected to various established surface treatments to increase surface area or surface roughness for better tissue integration or tissue attachment. Representative surface treatments include, but are not limited to, physical treatments and chemical treatments. Physical treatments include, e.g., machined process, sandblasting process, metallic deposition, non-metallic deposition (e.g., apatite deposition), or combinations thereof. Chemical treatment includes, e.g., etching using a chemical agent such as an acid, base (e.g., alkaline treatment), oxidation (e.g., heating oxidation and anodic oxidation), and combinations thereof. For example, a metallic medical implant can form different surface topographies by a machined process or an acid-etching process.

### Polymers

The polymers can be any polymer commonly used in the medical device industry. The polymers can be biocompatible or non-biocompatible. In some embodiments, the polymer can be poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), molecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof. Some examples of elastin protein mimetics include (LGGVG)ₙ, (VPGVG)ₙ, Val-Pro-Gly-Val-Gly, or synthetic biomimetic poly(L-glytanmate)-b-poly(2-acryloyloxyethyllactoside)-b-poly(1-glutamate) triblock copolymer.

In some embodiments, the polymer can be poly(ethylene-co-vinyl alcohol), poly(methoxyethyl methacrylate), poly(dihydroxylpropyl methacrylate), polymethacrylamide, aliphatic polyurethane, aromatic polyurethane, nitrocellulose, poly(ester amide benzyl), co-poly-{[N,N′-sebacoyl-bis-(L-leucine)-1,6-hexylene diester]_{0.75}-[N,N′-sebacoyl-L-lysine benzyl ester]_{0.25} (PEA-Bz), co-poly-{[N,N′-sebacoyl-bis-(L-leucine)-1,6-hexylene diester]_{0.75}-[N,N′-sebacoyl-L-lysine-4-amino-TEMPO amide]_{0.25}} (PEA-TEMPO), aliphatic polyester, aromatic polyester, fluorinated polymers such as poly(vinylidene fluoride-co-hexafluoropropylene), poly(vinylidene fluoride) (PVDF), and Teflon™ (polytetrafluoroethylene), a biopolymer such as elastin mimetic protein polymer, star or hyper-branched SIBS (styrene-block-isobutylene-block-styrene), or combinations thereof. In some embodiments, where the polymer is a copolymer, it can be a block copolymer that can be, e.g., di-, tri-, tetra-, or oligo-block copolymers or a random copolymer. In some embodiments, the polymer can also be branched polymers such as star polymers.

In some embodiments, a UV-transmitting material having the features described herein can exclude any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

### Medical Use

The medical implants provided herein can be used for treating, preventing, ameliorating, correcting, or reducing the symptoms of a medical condition by medical implanting the medical implants in a mammalian subject. The mammalian subject can be a human being or a veterinary animal such as a dog, a cat, a horse, a cow, a bull, or a monkey.

Representative medical conditions that can be treated or prevented using the medical implants provided herein include, but are not limited to, missing teeth or bone related medical conditions such as femoral neck fracture, missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a disorder or body condition such as, e.g., cancer, injury, systemic metabolism, infection or aging, and combinations thereof.

In some embodiments, the medical implants provided herein can be used to treat, prevent, ameliorate, or reduce symptoms of a medical condition such as missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a body condition or disorder such as cancer, injury, systemic metabolism, infection and aging, limb amputation resulting from injuries and diseases, and combinations thereof.

### EXAMPLES

The following examples illustrate, and shall not be construed to limit, the embodiments of the present invention.

### Example 1

### Studies on Activation of Medical Implant Materials

### SUMMARY

In the studies described in this example, we have shown that the temperature of medical implant materials significantly affect their capability to attract cells. Moreover, treating medical implant materials with UV light quickly and effectively recovers the temperature and more importantly significantly increase the once-decreased cell attraction capability. In the light of the above mentioned situation and concern of the current distribution and storage system of medical implant products, the currently used medical implant products are considered not to be in the optimal conditions in terms of the temperature and biological capability. Therefore, it is extremely important that the UV treatment is required immediately before their use to patients at the peripheral users' levels. We also demonstrated that to more effectively and rapidly recover the temperature and biological capability of medical implant materials, UV treatment should be performed in the closed condition, such as in a chamber without air flow. The UV treatment has proven to be effective to other medical implant materials than titanium, such as CoCr alloy.

However, UV light application to recover the temperature of medical implant materials and, more significantly, to regenerate their bioactivity is inventive. Also, the newly specified procedures of its use, i.e., the use immediately before the use of medical implant devices in chamber or similar closed conditions, are medically important for the procedures described herein would allow a medical practitioner to provide superior medical care pertaining to implantation, e.g., faster healing, stronger osseointegration of implant, etc. Because UV treatment is established as a safe, harmless, and inexpensive physicochemical treatment for metallic instruments, this technology will provide a significant technical advantage in its clinical and commercial application to enhance the currently used medical implant devices.

### Results.

### Cold Titanium Temperature Adversely Affects its Cell Attraction Capability

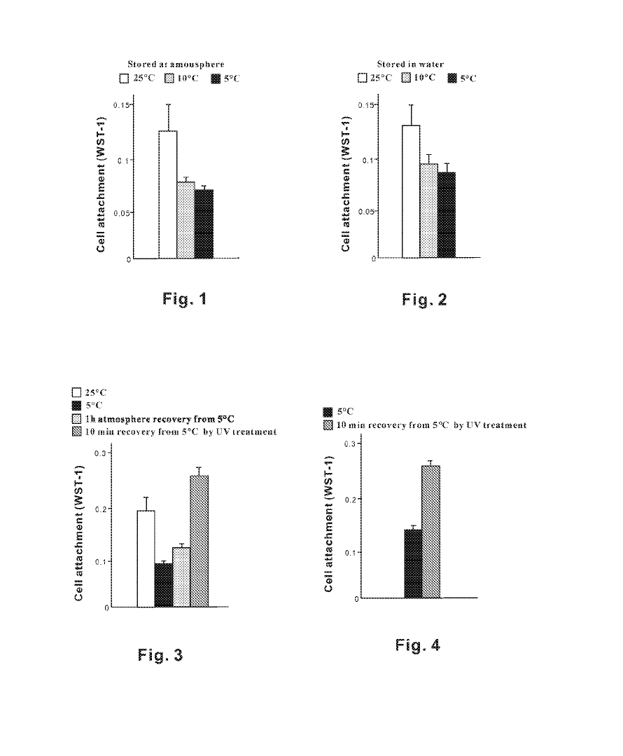

Three different titanium disks were prepared: 1) room temperature, stored in a 25° C. air for 3 h; 2) 10° C., stored in 10° C. air under sealed conditions for 3 h; and 3) 5° C., stored in 5° C. air for 3 h. Osteoblasts (bone-making cells) derived from rat bone marrow were seeded onto these disks. After 2 h of incubation, adhered cells were quantified using WST-1 assay (FIG. 1). The number of attached cells was significantly reduced on 5° C. and 10° C. titanium disks compared with the one on 25° C. disks (p>0.05), with the one on 5° C. disks being lowest. The reduction was as substantial as 45-50% from the level of 25° C. disks.

### Low Temperature Storage in Water Also Adversely Affects Cell Attraction Capability of Titanium

Three different titanium disks were prepared: 1) room temperature, stored in a 25° C. ddH₂O for 3 h; 2) 10° C., stored in 10° C. ddH₂O under sealed conditions for 3 h; and 3) 5° C., stored in 5° C. ddH₂O for 3 h. Osteoblasts were seeded onto these disks. After 2 h of incubation, adhered cells were quantified using WST-1 assay (FIG. 2). As shown in the results on the air experiments, the number of attached cells was significantly reduced on 5° C. and 10° C. titanium disks compared with the one on 25° C. disks (p>0.05), with the one on 5° C. disks being lowest. The reduction was as substantial as 35-40% from the level of 25° C. disks.

### UV Treatment of Cold Titanium Rapidly and Effectively Recovers its Temperature and Cell Attraction Capability

Four different titanium disks were prepared: 1) room temperature, stored in a 25° C. air for 3 h; 2) 5° C., stored in 5° C. air under sealed conditions for 3 h; 3) atmospheric recovery, 5° C. disks after being stored in room temperature air for 1 h; and 4) UV recovery, 5° C. disks after being treated with UV for 10 min. Osteoblasts were seeded onto these disks. After 3 h of incubation, adhered cells were quantified using WST-1 assay (FIG. 3). The number of attached cells was significantly lower on 5° C. disks than on 25° C. disks. The atmospheric recovery for 1 h increased the cell attachment by 25% (p<0.05) but the cell attachment was still significantly lower than the baseline level of 25° C. disks (p<0.05). UV treatment of 5° C. disks remarkably increased the lowered cell attachment to the level equivalent or even higher than 25° C. disks (p<0.05). The temperature of atmosphere-recovered disks and UV-recovered disks were 16.2° C. and 27.5° C., respectively.

### UV Treatment was Also Effective on CoCr Alloy to Recover its Temperature and Cell Attraction Capability

Two different CoCr alloy disks were prepared: 1) 5° C., stored in 5° C. air under sealed conditions for 3 h; 2) UV recovery, 5° C. disks after being treated with UV for 10 min. Osteoblasts were seeded onto these disks. After 3 h of incubation, adhered cells were quantified using WST-1 assay (FIG. 4). The number of cells attached to 5° C. disks was significantly elevated after treating the disks with UV by 50%.

UV Treatment of Cold Titanium is More Effective Under Closed Conditions than in Open Conditions

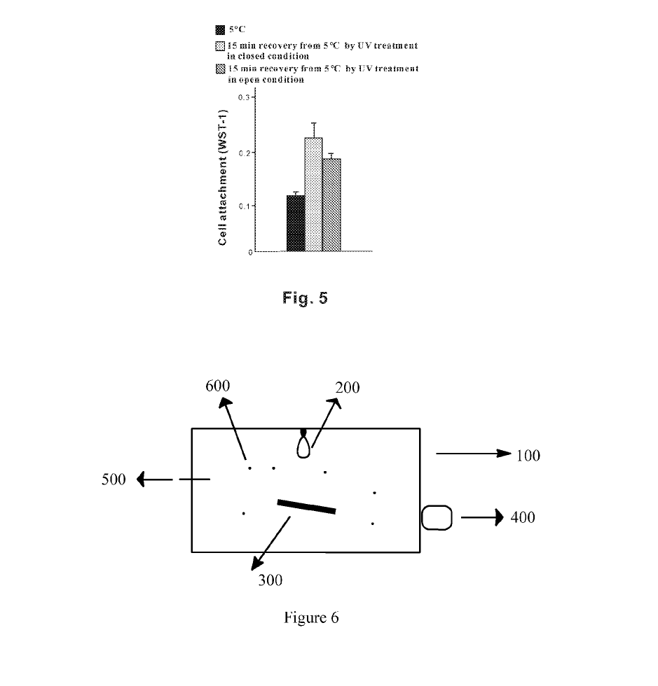

Three different titanium disks were prepared: 1) 5° C., stored in 5° C. air under sealed conditions for 3 h; 2) UV recovery under closed conditions, 5° C. disks after being treated with UV for 15 min in a closed chamber; and 3) UV recovery under open conditions, 5° C. disks after being treated with UV for 15 min in an open atmosphere. Osteoblasts were seeded onto these disks. After 3 h of incubation, adhered cells were quantified using WST-1 assay (FIG. 5). The number of attached cell was significantly recovered by both of the UV recovery protocols but the amount of the recover was significantly greater for the UV treatment in a closed chamber. The temperature of titanium disks after UV treatment in a closed chamber and open atmosphere was 26.5° C. and 17.2° C., respectively.

### Materials and Methods

### Titanium Sample

Disks (20 mm in diameter and 1.0 mm in thickness) made of commercially pure titanium (Grade 2) or CoCr alloy were used. Titanium disks were acid-etched with 67% H₂SO₄ at 120° C. for 75 seconds to simulate the most commonly used surface in the market. CoCr disks were used as machine-prepared. UV treatment was performed using UV light; intensity, ca. 0.5 mW/cm² (λ=360±20 nm) and 1.5 mW/cm² (λ=250±20 nm) for all experiments except for the chamber/open atmosphere experiment. UV light of ca. 2.0 mW/cm² (λ=360±20 nm) was used for the chamber/open atmosphere experiment. The temperature of the titanium disks was measured by surface thermometer (AD-5601A, AND Inc., Tokyo, Japan).

### Bone-Forming Cell (Osteoblast) Cell Culture

Bone marrow cells isolated from the femur of 8-week-old male Sprague-Dawley rats were placed into alpha-modified Eagle's medium supplemented with 15% fetal bovine serum, 50 mg/ml ascorbic acid, 10⁻⁸M dexamethasone, 10 mM Na-β-glycerophosphate and Antibiotic-antimycotic solution containing 10000 units/ml Penicillin G sodium, 10000 mg/ml Streptomycin sulfate and 25 mg/ml Amphotericin B. Cells were incubated in a humidified atmosphere of 95% air, 5% CO₂ at 37° C. At 80% confluency, the cells were detached using 0.25% Trypsin-1 mM EDTA-4 Na and seeded onto titanium disks at a density of 3×10⁴ cells/cm².

### Cell Attachment

Initial attachment of cells was evaluated by measuring the quantity of the cells attached to titanium substrates after 2 hours or 3 hours of incubation. The quantifications was performed using WST-1 based colorimetry (WST-1, Roche Applied Science, Mannnheim, Germany). The culture well was incubated at 37° C. for 4 hours with 100 tetrazolium salt (WST-1) reagent. The amount of formazan product was measured using an ELISA reader at 420 nm.

### Statistical Analysis

ANOVA was used to examine differences in variables between differently treated titanium disks. If necessary, a post-hoc Bonferroni test was used as a multiple comparisons test; p<0.05 was considered significant.

### REFERENCES

[1] Ray N F, Chan J K, Thamer M, Melton L J, 3rd. Medical expenditures for the treatment of osteoporotic fractures in the United States in 1995: report from the National Osteoporosis Foundation. J Bone Miner Res 1997; 12:24.
[2] Hudson J I, Kenzora J E, Hebel J R, Gardner J F, Scherlis L, Epstein R S, Magaziner J S. Eight-year outcome associated with clinical options in the management of femoral neck fractures. Clin Orthop Relat Res 1998:59.
[3] Lu-Yao G L, Keller R B, Littenberg B, Wennberg J E. Outcomes after displaced fractures of the femoral neck. A meta-analysis of one hundred and six published reports. J Bone Joint Surg Am 1994; 76:15.
[4] Tidermark J, Ponzer S, Svensson O, Soderqvist A, Tornkvist H. Internal fixation compared with total hip replacement for displaced femoral neck fractures in the elderly. A randomised, controlled trial. J Bone Joint Surg Br 2003; 85:380.
[5] Ravikumar K J, Marsh G. Internal fixation versus hemiarthroplasty versus total hip arthroplasty for displaced subcapital fractures of femur—13 year results of a prospective randomised study. Injury 2000; 31:793.
[6] van Steenberghe D, Jacobs R, Desnyder M, Maffei G, Quirynen M. The relative impact of local and endogenous patient-related factors on medical implant failure up to the abutment stage. Clin Oral Medical implants Res 2002; 13:617.
[7] Ozawa S, Ogawa T, Iida K, Sukotjo C, Hasegawa H, Nishimura R D, Nishimura 1. Ovariectomy hinders the early stage of bone-medical implant integration: histomorphometric, biomechanical, and molecular analyses. Bone 2002; 30:137.
[8] Nevins M L, Karimbux N Y, Weber H P, Giannobile W V, Fiorellini J P. Wound healing around endosseous medical implants in experimental diabetes. Int J Oral Maxillofac Medical implants 1998; 13:620.
[9] Zhang H, Lewis C G, Aronow M S, Gronowicz G A. The effects of patient age on human osteoblasts' response to Ti-6Al-4V medical implants in vitro. J Orthop Res 2004; 22:30.
[10] Takeshita F, Murai K, Ayukawa Y, Suetsugu T. Effects of aging on titanium medical implants inserted into the tibiae of female rats using light microscopy, SEM, and image processing. J Biomed Mater Res 1997; 34:1.
[11] Yamazaki M, Shirota T, Tokugawa Y, Motohashi M, Ohno K, Michi K, Yamaguchi A. Bone reactions to titanium screw medical implants in ovariectomized animals. Oral Surg Oral Med Oral Pathol Oral Radiol Endod 1999; 87:411.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method of fabricating a medical implant, comprising
treating a medical implant with ultraviolet light (UV) in a closed environment, and
causing the temperature of the medical implant to be between room temperature (Rt) and about 37° C.,
wherein the treating and causing acts are performed immediately prior to placing the medical implant in a site in need thereof in a subject.

2. The method of claim 1, wherein the medical implant has a temperature or exposed to a temperature below Rt or above body temperature, prior to the UV treatment.

3. The method according to claim 1, wherein the medical implant has a temperature or is exposed to a temperature between 0° C. and about 20° C., prior to receiving the UV treatment.

4. The method according to claim 1, wherein the medical implant has a temperature or is exposed to a temperature 40° C. or above, prior to receiving the UV treatment.

5. The method of claim 1, wherein the closed environment is a closed chamber.

6. The method of claim 1, wherein the closed environment is a closed chamber filled with an inert gas, clean air, or carbon-free air.

7. The method of claim 6, wherein the inert gas comprises N₂, He, or Ar.

8. The method of claim 1, wherein the medical implant comprises a metallic material.

9. The method of claim 1, wherein medical implant comprises a surface comprising a microstructure or a nanostructure.

10. The method of claim 7, wherein the metallic material comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

11. The method of claim 10, wherein the medical implant is selected from the group consisting of tooth medical implants, jaw bone medical implant, repairing and stabilizing screws, pins, frames, and plates for bone, spinal medical implants, femoral medical implants, neck medical implants, knee medical implants, wrist medical implants, joint medical implants such as an artificial hip joint, maxillofacial medical implants such as ear and nose medical implants, limb prostheses for conditions resulting from injury and disease, and combinations thereof.

12. The method of claim 1, wherein the medical implant comprises a non-metallic material.

13. The method of claim 12, wherein the non-metallic material comprises a polymeric material or a bone cement material.

14. The method of claim 13, wherein the bone cement material comprises a material selected from the group consisting of polyacrylates, polyesters, bioglass, ceramics, calcium-based materials, calcium phosphate-based materials, and combinations thereof.

15. The method of claim 13, wherein the bone cement material comprises poly(methyl methacrylate) (PMMA) or methyl methacrylate (MMA).

16. A system, comprising a chamber element, an ultraviolet light (UV) element, and a medical implant element;
wherein the chamber element forms a closed environment that houses the medical implant element and the UV element,
wherein the medical implant element comprises a medical implant having a temperature or being exposed to a temperature of below room temperature (Rt) or above body temperature, and
wherein, in the chamber element, the medical implant receives UV treatment and is caused to have a temperature between the room temperature and about 37° C.

17. The system according to claim 16, further comprising a timing element and a thermometer.

18. The system according to claim 16, further comprising an inert gas.

19. The system according to claim 16, wherein the medical implant is selected from the group consisting of tooth medical implants, jaw bone medical implant, repairing and stabilizing screws, pins, frames, and plates for bone, spinal medical implants, femoral medical implants, neck medical implants, knee medical implants, wrist medical implants, joint medical implants, an artificial hip joint, maxillofacial medical implants, ear implants, nose medical implants, limb prostheses for conditions resulting from injury and disease, and combinations thereof.

20. The system according to claim 16, wherein the medical Implant has a temperature or is exposed to a temperature between 0° C. and about 20° C., prior to receiving the UV treatment.

21. The system according to claim 15, wherein the medical implant has a temperature or is exposed to a temperature about 40° C. or above, prior to receiving the UV treatment.
